(19) United States
(12) Patent Application Publication
(10) Pub. No.: US 2024/0382719 A1
(43) Pub. Date: Nov. 21, 2024

(54) High-Pressure Balloon for a Catheter and Method of Manufacture

(71) Applicant: Intervention Tech LLC, Irvine, CA (US)

(72) Inventor: Michael Douglas Franklin, JR., Irvine, CA (US)

(73) Assignee: Intervention Tech LLC, Irvine, CA (US)

(21) Appl. No.: 18/199,941

(22) Filed: May 20, 2023

(51) Int. Cl. A61M 25/10

(52) U.S. Cl. CPC A61M 25/1029; A61M 2025/1031; A61M 2025/1084

(57) ABSTRACT

A balloon catheter has a shaft having a Y-connector provided at the proximal end, and a balloon provided at the distal end, the balloon having a balloon body having opposing first and second ends, and a reinforcement fiber layer wrapped on the balloon body, the fiber layer formed from a single continuous fiber wrapped first radially around the balloon body in non-continuous rows, followed by a figure-8 wrap that continuously traverses from the first end to the second end, and the second end back to the first end.

### BACKGROUND OF THE INVENTION

### 1. Field of the Invention

The present invention relates to catheters, and in particular to a high-pressure balloon for a catheter that is adapted to fracture implanted surgical heart valves, and methods for manufacturing the balloon.

### 2. Description of the Prior Art

In surgical and transcatheter heart valves, there are different bioprosthetic implants sold with various internal designs and structures. These structures can consist of various polymers and/or metal (i.e. stainless steel, nitinol, cobalt chromium etc.) “frames” or “rings” which provide support for the other valve components such as textile sewing rings, commissure posts and tissue leaflets. In all cases, the heart valve can be expanded some amount by a high-pressure balloon to cause fracture (fracking) of the internal structures or in other cases bending/deforming some element of the structure to increase the effective overall internal diameter, so as to allow a new transcatheter heart valve to be implanted at the same location.

Existing high-pressure balloons sometimes used for these applications include the Bard True Balloon™ and Bard Atlas Gold™. These balloons can sometimes achieve the pressures needed, although their rated burst pressure (RBP) is often below the required pressure. There are other examples of fiber-reinforced high pressure balloons in use today, including the Boston Scientific Athletis™ balloon; however this balloon lacks the appropriate sizes and strength as well as other potential drawbacks.

### SUMMARY OF THE DISCLOSURE

It is an object of the present invention to provide a high-pressure balloon for use in a catheter that can be adapted to effectively frack an implanted surgical heart valve.

It is another object of the present invention to provide a balloon catheter that is intended to be used to allow needed treatment of structural heart disease patients with conditions requiring a higher-pressure balloon than existing products on the market.

To meet the objectives of the present invention, there is provided a balloon catheter having a main shaft with a Y-connector provided at the proximal end, and a balloon provided at the distal end, the balloon having a balloon body having opposing first and second ends, and a reinforcement fiber layer wrapped on the balloon body, the fiber layer formed from a single continuous fiber wrapped first radially around the balloon body in non-continuous rows, followed by a figure-8 wrap that continuously traverses from the first end to the second end, and the second end back to the first end.

The balloon catheter of the present invention can be manufactured according to the following steps. First, a shaft is provided having a distal end and a proximal end, a Y-connector provided at the proximal end, and a balloon provided at the distal end, the balloon having a balloon body having a cylindrical central section having first and second ends, a first tapered neck at the first end, a second tapered neck at the second end, a first cone at an end of the first tapered end, and a second cone at an end of the second tapered end. Next, a single fiber is wrapped in a radial wrap that extends from the first tapered neck and the first end across the cylindrical central section to the second end, and then traversing from the second end back to the first wrap. Next, at the end of the radial wrap, the single fiber is wrapped in a figure-8 wrap that traverses opposite locations of the first and second tapered neck sections, wherein the single fiber extends from a first location on the first tapered neck and extends across the cylindrical central section to a second location on the second tapered neck, and from the second location, extends back across the cylindrical central section to a third location on the first tapered neck that is spaced apart from the first location, and from the third location, extends back across the cylindrical central section to a fourth location on the second tapered neck that is spaced apart from the second location, and so on.

In addition to fracking surgical heart valves, other indications have been identified where the balloon according to the present invention can be beneficially used. These include but are not limited to:
1. Transcatheter Valve-in-Valve (ViV) where a previously implanted transcatheter valve has diminished functionality and it is desirable to expand it to allow a new THV (transcatheter heart valve) implantation with optimized EOA (Effective Orifice Area). These are typically aortic or pulmonary valves, but could also be mitral or tricuspid valves, depending on size.
2. Heavily calcified anatomy and structures where a hard/non-compliant ring of calcium has formed in a radial configuration requiring a non-compliant balloon catheter to avoid over-expansion. This can include native tissue valves as well as other bioprosthetics, such as pulmonary conduits, annular patches or stents implanted during prior therapies.

### BRIEF DESCRIPTION OF THE DRAWINGS

FIG. 1 is a schematic view of a balloon catheter according to one embodiment of the present invention.

FIG. 2A is a schematic view of the balloon of the catheter of FIG. 1 showing the first pass of the radial wrap step in the method of manufacture for the balloon.

FIG. 2B is a schematic view of the balloon of the catheter of FIG. 1 showing the second pass of the radial wrap step in the method of manufacture for the balloon.

FIG. 3A and FIG. 3B are the front views for the balloon in FIGS. 2A and 2B, respectively.

FIGS. 4A-4B are the perspective views for the balloon in FIGS. 2A and 2B, respectively.

FIG. 5A is a schematic view of the balloon of the catheter of FIG. 1 showing the first pass of the Figure-8 wrap step in the method of manufacture for the balloon.

FIG. 5B is a schematic view of the balloon of the catheter of FIG. 1 showing the second pass of the Figure-8 wrap step in the method of manufacture for the balloon.

FIG. 5C is a schematic view of the balloon of the catheter of FIG. 1 showing the third pass of the Figure-8 wrap step in the method of manufacture for the balloon.

FIGS. 6A-6C are the front views for the balloon in FIGS. 5A-5C, respectively.

FIGS. 7A-7C are the perspective views for the balloon in FIGS. 5A-5C, respectively.

FIG. 8 is an enlarged cross-sectional view of a portion of the balloon in FIG. 1.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The present invention provides a high-pressure balloon that is a hybrid design between traditional blow molded medical balloons and purely constructed composite materials (i.e., the Bard True™ balloon). As a result, it incorporates strengths and benefits of both concepts by using a unique and novel method of manufacturing. Typically, when reinforcement fibers are used, they are formed using a braided, woven or knit structure formed by a machine and then transferred or directly applied to the balloon surface or other materials used in construction. The density and number of carriers of fiber for the strength generally results in an excessively large profile at the balloon cones. The present invention uses a single strand of fiber that is wrapped in a manner that applies structural support only where primarily needed and results in sufficient radial and axial support for the pressures required.

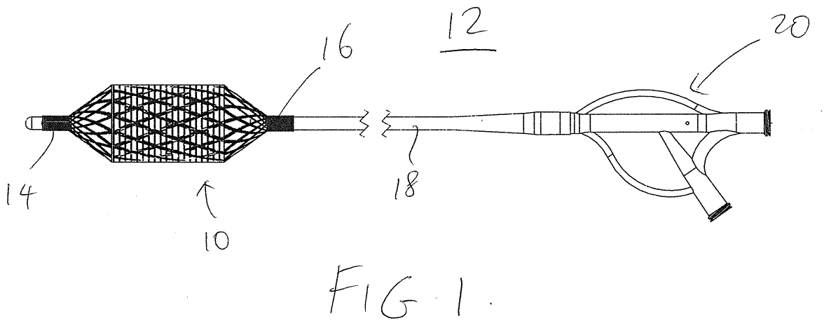

Referring to FIG. 1, the present invention provides a high-pressure balloon 10 on a catheter 12. The balloon 10 has two opposing cones 14 and 16 provided along the shaft 18 of the catheter 12. The catheter 12 includes a Y-connector 20. The construction of the catheter 12 is not critical to the balloon 10 and can be embodied in the form of any conventional over the wire or rapid exchange balloon catheter, so additional details of the catheter 12 will not be described.

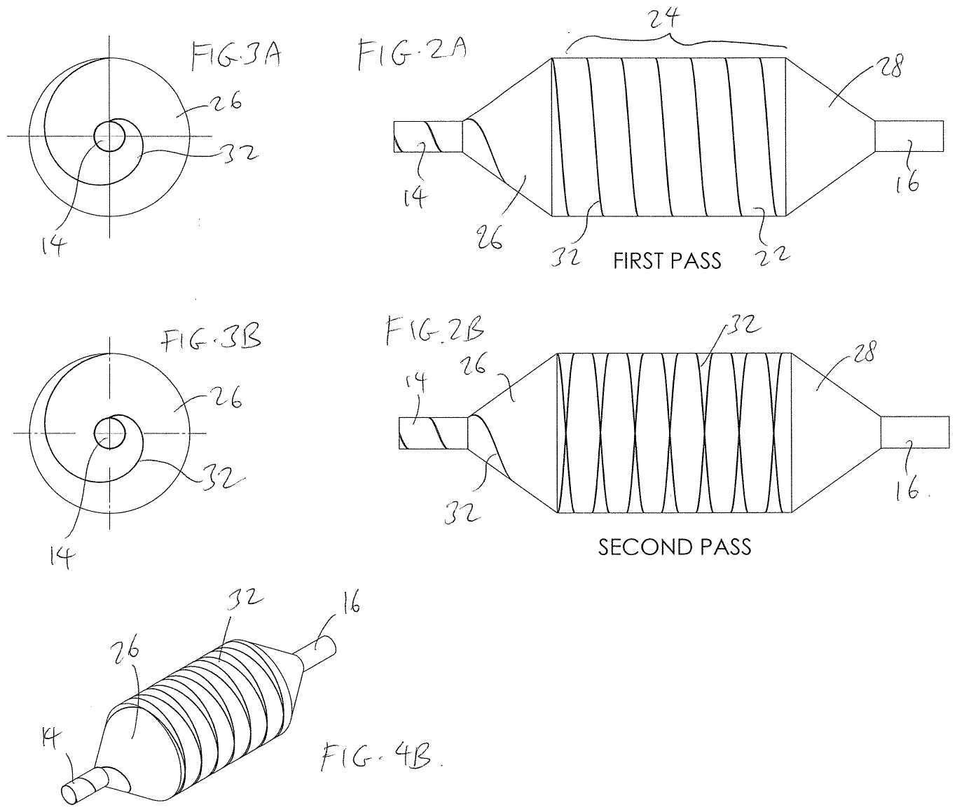

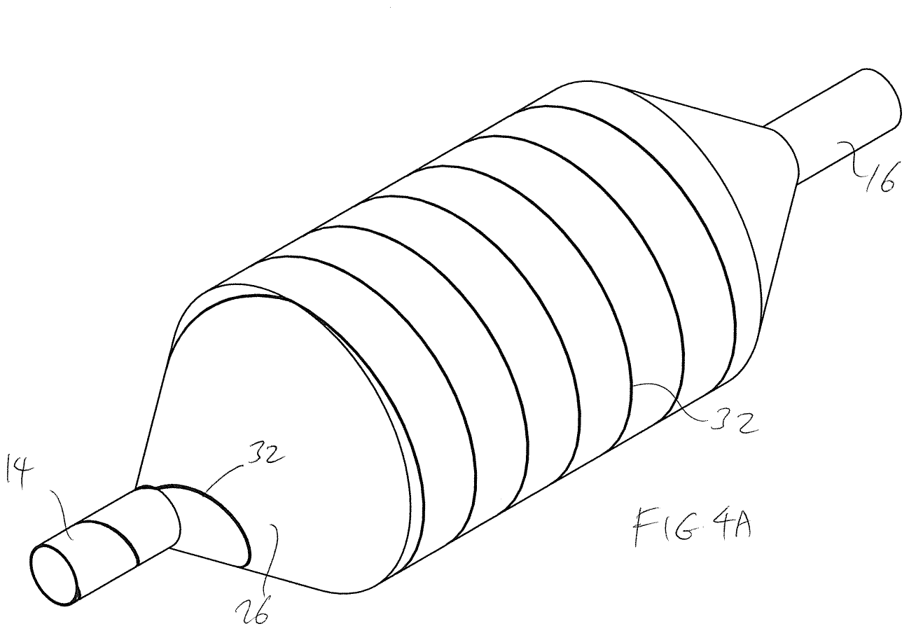

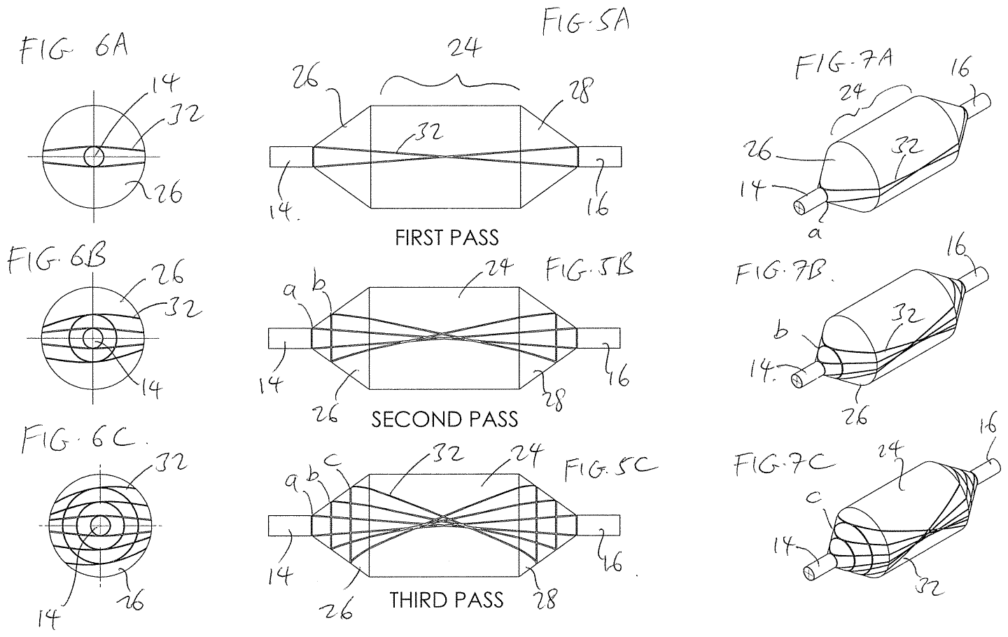

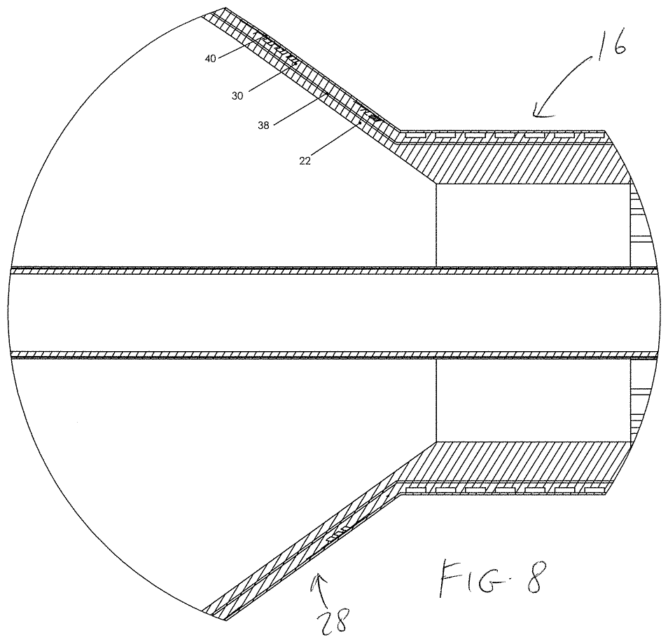

The high-pressure balloon 10 of the present invention preferably includes four primary materials/layers (see FIG. 8):
1. A conventional thin-wall blow-molded semi-compliant balloon body 22. This is currently a Nylon 12 material (e.g., Vestamid Care™ ML21) but could potentially be made from PET, PEBAX, or other medical grade polymer. This balloon body 22 can have a generally cylindrical central section 24 and opposing tapered necks 26 and 28. The balloon body 22 acts as a liner for sealing pressure and as a forming mandrel on which additional layers are formed.
2. A base coating of a tacky or non-lubricious material 38 is applied to the Nylon 12 balloon. This is preferably a Kraton coating but could be another medical grade polymer. There are also surface treatments that can be performed to make the outer Nylon surface non-lubricious; such surface treatments are well-known in the art and are not described in greater detail herein. The purpose of this base coating is to make the surface of the balloon layer tacky and create the needed friction for the reinforcing fibers to stay in the appropriate locations.
3. A Dyneema radial reinforcement fiber layer 30 (see FIGS. 2A-4) formed from a single continuous fiber wrapped first radially around the balloon's diameter in non-continuous rows (i.e., the space between each radial wrap that is filled in by subsequent passes in alternating directions) until adequate strength is achieved. The continuous fiber then changes pattern to a “figure-8” style axial/longitudinal support (see FIGS. 5A-7C) to an axial looping wrap which radially partial-wraps the cones and necks of the balloon while traversing from end to end continuously to reinforce the balloon's length. The reinforcing fiber layer is preferably an “Ultra High Molecular Weight Polyethylene” (UHMWPE) but can be another strong fiber. The final balloon 10 is shown in FIG. 1.
4. An encapsulating layer 40 of medical grade polymer to hold the reinforcement fiber layer together and prevent fiber migration while providing a smooth outer surface. This encapsulating layer 40 is preferably a polycarbonate-urethane material (PCU) but could be another medical grade polymer.

FIGS. 2A to 7C illustrate the method for manufacturing the balloon 10. In the first step, the base balloon body 22 is blow molded on a balloon forming machine, and then later assembled on the catheter. The initial size molds range from 19 mm to 30 mm in 1 mm increments.

In the second step, the base coating polymer (e.g., Kraton FG1901 or other) is applied to the balloon body 22 using a solvent solution dip coating process as is well-known in the art.

In the third step, the single reinforcement fiber layer 30 is applied using a two-axis movement. A motor spins an inflated balloon while the operator or a second motor traverses the fiber back and forth along the length of the balloon. The single reinforcement fiber layer 30 is applied first by a radial wrap method, and then by an axial/longitudinal figure-8 wrap method.

FIGS. 2A-4 illustrate the radial wrap method. The fiber layer 30 has a single fiber 32. The single fiber 32 has an initial entry path at the cone 14 and is radially wrapped around the tapered neck 26 and the central section 24 for a first pass. See FIGS. 2A, 3A and 4. The wrapping does not extend into the tapered neck 28 because radial reinforcement of both cones is not required during this step. At the end of the central section 24 adjacent the tapered neck 28, the wrapping reverses direction and returns towards the tapered neck 26 for the second pass. See FIG. 2B. On the second pass, the wrap ends at the end of the central section 24 and does not extend into the tapered neck 26. The completion of a first pass and a second pass constitutes one cycle. The radial wrapping then proceeds for another first pass and second pass to complete a second cycle. The process continues until the full radial wrap process is completed.

FIGS. 5A-7C illustrate the figure-8 wrap method. To simplify these drawings, these drawings do not show the underlying radial wrap that has already been completed. This figure-8 wrapping essentially traverses the opposite general location on the balloon body 22. This traverse is what places the lengthwise or longitudinal fiber sections into a figure-8 configuration. At the start of figure-8 wrap, the fiber 32 from the end of the radial wrap can begin from the largest-diameter section of the cone 14, which can be adjacent or immediately next to the smallest-diameter portion of the tapered neck 26. The fiber 32 traverses the tapered neck 26 across the central section 24 and the tapered neck 28, then wraps around the other cone 16 at the location where the cone 16 is adjacent or immediately next to the smallest-diameter portion of the tapered neck 28. The fiber 32 then traverses the tapered neck 28 and back across the central section 24 and the tapered neck 26. This completes a first pass as shown in FIGS. 5A, 6A and 7A. The second pass can begin at a new location along the tapered neck 26 (see FIG. 5B) and traverse to a corresponding opposite location at the tapered neck 28 before traversing back to the tapered neck 26. The third pass can begin at another new location along the tapered neck 26 (see FIG. 5C) and traverse to a corresponding opposite location at the tapered neck 28 before traversing back to the tapered neck 26. FIGS. 5A-5C show the traverse decreasing in distance along each successive pass, but it is also possible to have the FIG. 8 wrapping begin along the tapered necks 26 and 28 close to the central section 24 so that the traverse increases in distance along each successive pass.

The final wrapped balloon 10 is shown in FIG. 1 and has a generally uniform distribution along the entire length and around the circumference of the balloon 10.

Although the method of the present invention provides the radial wrap before the figure-8 wrap, it is also possible to perform the figure-8 wrap before the radial wrap.

In the fourth step, the outer coating of encapsulating layer 40 of medical grade polymer is applied in the form of a PolyCarbonate-Urethane (PCU Carbothane 3585A) or similar material dip coated with a polymer/solvent solution. This layer 40 encapsulates the fiber 32 and locks the fiber wrap 30 into their positions and provides a smooth outer surface layer.

The method of the present invention is designed to provide adequate strength to the balloon, an optimized pleated/folded profile while minimizing manufacturing challenges and costs faced by more complex methods such as braiding, weaving or otherwise attaching a textile construct to augment or function as a balloon. The radial wrap is placed in non-continuous rows to prevent localized failure. This can be described as one or more sections of the fiber breaking which if continuously applied would result in the adjacent radial fibers to become loosened or weakened due to being unsupported. The figure-8 wrap is designed to provide both a partial radial wrap supporting the conical neck sections while also anchoring the fiber during axial traverses to support the longitudinal section of the balloon and prevent length compliance/stretching.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A balloon catheter, comprising:
a shaft having a distal end and a proximal end;
a Y-connector provided at the proximal end; and
a balloon provided at the distal end, the balloon having a balloon body having opposing first and second ends, and a reinforcement fiber layer wrapped on the balloon body, the fiber layer formed from a single continuous fiber wrapped first radially around the balloon body in non-continuous rows, followed by a figure-8 wrap that continuously traverses from the first end to the second end, and the second end back to the first end.

2. The catheter of claim 1, wherein the balloon body has an outer surface and a base coating of tacky material is applied to the outer surface of balloon body.

3. The catheter of claim 2, wherein the base coating of tacky material is Kraton.

4. The catheter of claim 2, wherein the reinforcement fiber layer is wrapped over the balloon body and the base coating.

5. The catheter of claim 1, wherein an encapsulating polymer layer is applied over the reinforcement fiber layer.

6. The catheter of claim 4, wherein an encapsulating polymer layer is applied over the reinforcement fiber layer.

7. The catheter of claim 1, wherein the balloon body has a cylindrical central section having opposite ends, and a tapered neck provided at each of the opposite ends, and wherein the radial wrap does not cover at one of the tapered necks.

8. The catheter of claim 1, wherein the balloon body has a cylindrical central section having opposite ends, and a tapered neck provided at each of the opposite ends, and wherein the figure-8 wrap extends across the cylindrical central section and the tapered necks.

9. A method of making a balloon for a balloon catheter, comprising:
providing a shaft having a distal end and a proximal end, a Y-connector provided at the proximal end, and a balloon provided at the distal end, the balloon having a balloon body having a cylindrical central section having first and second ends, a first tapered neck at the first end, a second tapered neck at the second end, a first cone at an end of the first tapered end, and a second cone at an end of the second tapered end;
wrapping a single fiber in a radial wrap that extends from the first tapered neck and the first end across the cylindrical central section to the second end, and then traversing from the second end back to the first wrap; and
at the end of the radial wrap, wrapping the single fiber in a figure-8 wrap that traverses opposite locations of the first and second tapered neck sections, wherein the single fiber extends from a first location on the first tapered neck and extends across the cylindrical central section to a second location on the second tapered neck, and from the second location, extends back across the cylindrical central section to a third location on the first tapered neck that is spaced apart from the first location, and from the third location, extends back across the cylindrical central section to a fourth location on the second tapered neck that is spaced apart from the second location, and so on.

10. The method of claim 9, wherein the balloon body has an outer surface, further including the step of applying a base coating of tacky material to the outer surface of balloon body before the wrapping a single fiber in a radial wrap step.

11. The method of claim 10, further including the step of applying an encapsulating polymer layer over the single fiber after wrapping the single fiber in a figure-8 wrap step.
